(19) United States
(12) Patent Application Publication
(10) Pub. No.: US 2024/0202055 A1
(43) Pub. Date: Jun. 20, 2024

(54) MISSION-CENTRIC HEALTH WARNING SYSTEM FOR HIGH VALUE ASSETS

(71) Applicant: Raytheon Company, Waltham, MA (US)

(72) Inventors: John A. Cogliandro, Dedham, MA (US); Jennifer L. Pueschner, Oro Valley, AZ (US); Darcie R. Volk, North Andover, MA (US); Brian Gin, Vail, AZ (US); Emile Mark Szlemko, Vail, AZ (US); Oran Dale May, Tucson, AZ (US); Stephen Lester Fecteau, Amherst, MA (US)

(73) Assignee: Raytheon Company, Waltham, MA (US)

(21) Appl. No.: 18/176,769

(22) Filed: Mar. 1, 2023

Related U.S. Application Data
(60) Provisional application No. 63/337,294, filed on May 2, 2022.
(60) Provisional application No. 63/337,297, filed on May 2, 2022.

(51) Int. Cl. G06F 11/00; G06F 11/07; G06Q 50/26

(52) U.S. Cl. CPC G06F 11/004; G06F 11/0736; G06F 11/076; G06F 11/0772; G06Q 50/26

(57) ABSTRACT

A method for use in a computing device, including: receiving a first value of a first parameter, the first value being generated by a monitoring device that is arranged to monitor a respective monitored system; retrieving, from a memory, a first threshold that is associated with the first parameter and the respective monitored system; and generating a first system condition alert when the first value has crossed the first threshold.

### CROSS-REFERENCE TO RELATED APPLICATIONS

This application claims benefit to U.S. Patent Application No. 63/337,294, filed May 2, 2022 and entitled “High Value Asset—Mission Centric Health Warning System”, the entire contents of which are incorporated herein by reference. This application claims benefit to U.S. Patent Application No. 63/337,297, filed May 2, 2022 and entitled “High Value Asset/Automated Weapon Health Active Monitoring” the entire contents of which are incorporated herein by reference.

### BACKGROUND

Providing strategic prognostics and system health related information for defense systems, such as effectors (also known as missiles) is a demand from many defense customers, such as the Army, Navy, or Air Force. Defense customers need immediate “mission centric” feedback to determine the system's relative readiness for a mission at a point in time. Such information can be used to enable prognostic development for assets that previously had no sustainment capabilities.

### SUMMARY

According to aspects of the disclosure, a method for use in a computing device, the method comprising: receiving a first value of a first parameter, the first value being generated by a monitoring device that is arranged to monitor a respective monitored system; retrieving, from a memory, a first threshold that is associated with the first parameter and the respective monitored system; and returning a first system condition alert when the first value has crossed the first threshold.

According to aspects of the disclosure, a system is provided, comprising: a memory; and at least one processor operatively coupled to the memory, the at least one processor being configured to perform the operations of: receiving a first value of a first parameter, the first value being received from a monitoring device that is arranged to monitor a respective monitored system; retrieving, from the memory, a first threshold that is associated with the first parameter and the respective monitored system; and generating a first system condition alert when the first value has crossed the first threshold.

According to aspects of the disclosure, a method is provided, comprising: obtaining a mission identifier, the mission identifier specifying a mission for a platform that is arranged to carry a monitored system; and selecting a threshold value for a threshold, the threshold corresponding to a monitored parameter of the monitored system, the threshold value being selected based on the mission identifier; and configuring at least one of a notification device and a data assessment system to generate an alert when the monitored parameter has crossed the threshold value.

### BRIEF DESCRIPTION OF THE DRAWINGS

The foregoing features may be more fully understood from the following description of the drawings in which:

FIG. 1A is a diagram of a missile launching system 100A, according to aspects of the disclosure;

FIG. 1B is a diagram of an example of a system, according to aspects of the disclosure;

FIG. 1C is a diagram of an example of a monitoring device, according to aspects of the disclosure;

FIG. 1D is a perspective view of a monitoring devices, according to aspects of the disclosure;

FIG. 1E is a diagram of an example of a Landsmont box, according to aspects of the disclosure;

FIG. 1F is a diagram of an example of a system, according to aspects of the disclosure;

FIG. 2A is a diagram of an example of a data assessment system, according to aspects of the disclosure;

FIG. 2B is a diagram of an example of a registration database, according to aspects of the disclosure;

FIG. 2C is a diagram of an example of an active thresholds database, according to aspects of the disclosure;

FIG. 2D is a diagram of an example of a mission-specific thresholds database 208, according to aspects of the disclosure;

FIG. 2E is a diagram of an example of a record, according to aspects of the disclosure;

FIG. 3A is a flowchart of an example of a process, according to aspects of the disclosure;

FIG. 3B is a flowchart of an example of a process, according to aspects of the disclosure;

FIG. 3C is a flowchart of an example of a process, according to aspects of the disclosure;

FIG. 3D is a flowchart of an example of a process, according to aspects of the disclosure;

FIG. 3E is a plot of possible values of parameters that are being monitored by a monitoring device, according to aspects of the disclosure;

FIG. 3F is a plot of possible values of a parameter that is being measured by a monitoring device, according to aspects of the disclosure;

FIG. 4A is a diagram illustrating an example of a use case for a monitoring device, according to aspects of the disclosure;

FIG. 4B is a diagram of respective threshold sets, according to aspects of the disclosure;

FIG. 4C is a diagram of a notification device, according to aspects of the disclosure;

FIG. 4D is a diagram of an example of a display unit, according to aspects of the disclosure;

FIG. 4E is a diagram of an example of a display unit, according to aspects of the disclosure;

FIG. 4F is a diagram of an example of a display unit, according to aspects of the disclosure;

FIG. 4G is a flowchart of an example of a process, according to aspects of the disclosure; and

FIG. 5 is a diagram of an example of a computing device, according to aspects of the disclosure.

### DETAILED DESCRIPTION

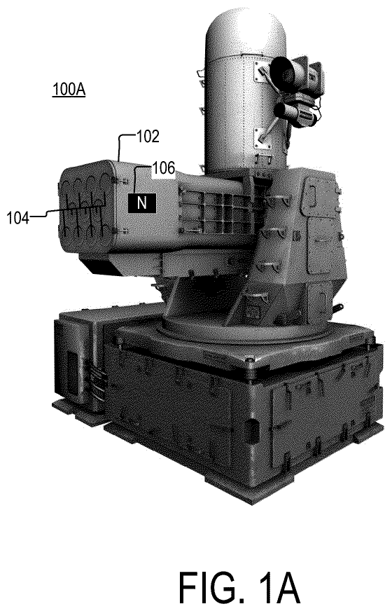

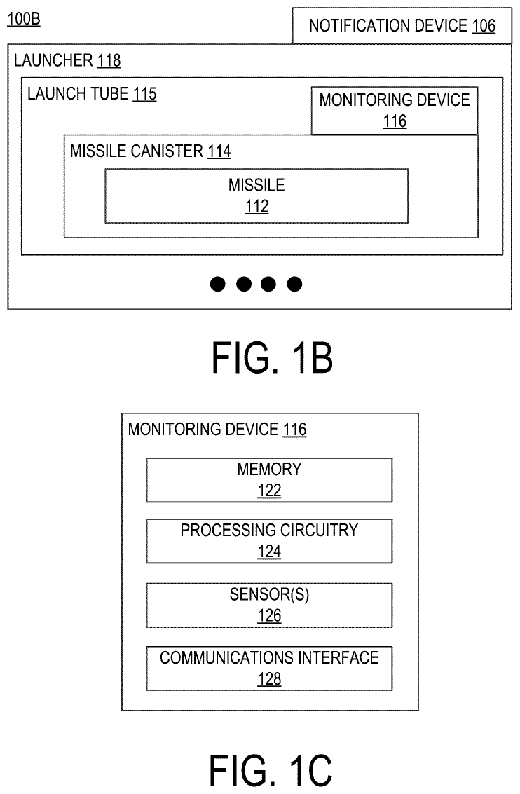

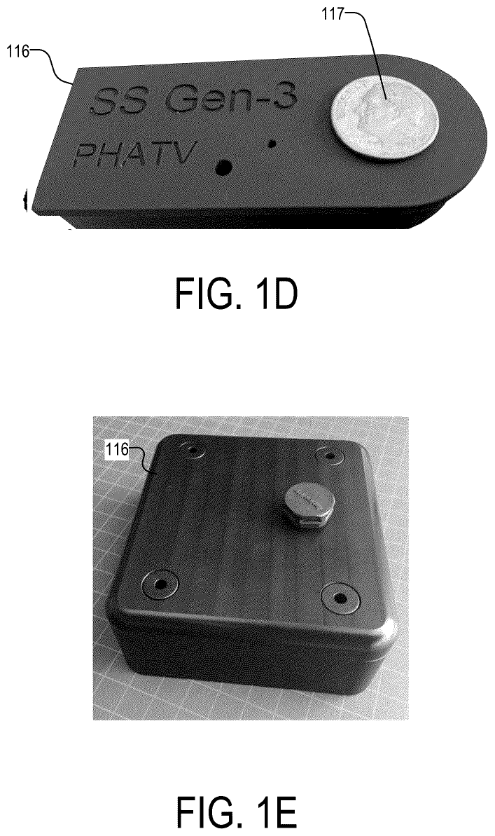

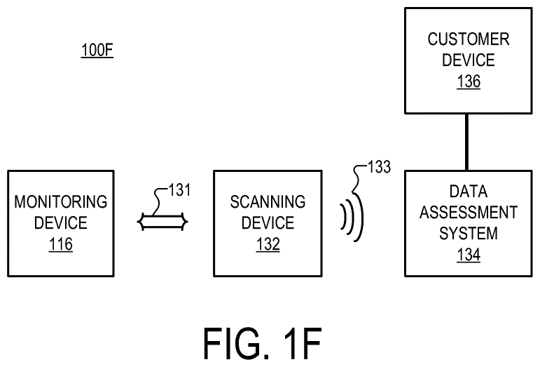

FIG. 1A is a diagram of a missile launching system 100A, according to aspects of the disclosure. As illustrated, the system 100A may include a missile launcher 102 having a plurality of launch tubes 104. Mounted on the exterior surface of the missile launcher 102 may be a notification device 106. The notification device 106 may be operatively coupled to one or more monitoring devices that are disposed inside the launcher 102. When any of the monitoring devices indicates that a missile in the launcher 102 has been exposed to excessive temperature or stress (e.g., pressure, vibration, shock or other system damaging occurrence), the notification device 106 may display an alert, notifying military personnel of the exposure.

FIG. 1B is a diagram of a system 100B, according to aspects of the disclosure. As illustrated, the system 100B may include a launcher 118 having one or more launch tubes 115. Inside each launch tube 115, there may be disposed a single or plurality of respective missile canisters 114. Inside each missile canister 114, there may be a respective missile 112. Mounted on an exterior surface of at least one missile canister may be a monitoring device 116. The monitoring device 116 may be paired with a notification device 106, which is mounted on the exterior surface of the launcher 118. Although in the example of FIG. 1B the monitoring device 116 and notification device 106 are used to monitor a missile, alternative implementations are possible in which the monitoring device 106 are used to monitor the launcher itself or an aircraft, a watercraft, a radar, a combat electronics system, and/or any other suitable device or system.

FIG. 1C is a diagram of an example of the monitoring device 116, according to aspects of the disclosure. As illustrated, the monitoring device 116 may include a memory 122, a processing circuitry 124, one or more sensors 126, and a communications interface 128. The memory 122 may include one or more of an electronically-erasable programmable read-only memory (EEPROM), a dynamic random-access memory, and/or any other suitable type of volatile or non-volatile memory. The processing circuitry 124 may include one or more of a general-purpose processor, an application-specific integrated circuit (ASIC), a Field-Programmable Gate Array (FPGA), and/or any other suitable type of processing circuitry. The sensors 126 may include one or more of a temperature sensor, a pressure sensor, an accelerometer, and/or any other suitable type of sensor. According to the present example, the communications interface 128 includes a radio frequency or magnetic communications interface. In some implementations, the communications interface may include a RuBee interface, which is marketed by Visible Assets, Inc. of Stratham, NH. However, alternative implementations are possible in which the communications interface 128 includes any suitable type of wired or wireless interface (e.g., a universal serial bus (USB) interface, a near-field communications (NFC) interface, a Bluetooth interface, etc.).

As is discussed further below, in operation, the processing circuitry 124 may use the sensors 126 to capture measurements of different system health parameters, and provide the measurements to a scanning device 132 (shown in FIG. 1F) and/or the notification device 106. For example, the processing circuitry 124 may be configured to take temperature measurements and provide the temperature measurements to the scanning device 132, when the scanning device 132 establishes a connection with the monitoring device 116. As another example, the processing circuitry 124 may be configured to take acceleration measurements, and provide the acceleration measurements to the scanning device 132 when the scanning device establishes a connection with the monitoring device 116. As yet another example, the monitoring device 116 may provide the temperature and/or acceleration measurements to the notification device 106. In another example, the external interface may have symbols or other warning lights that provide rudimentary system health information to a human without the use of a reader.

A non-limiting example of the utility of the monitoring device 116 is now provided in further detail. In general, a missile includes various sensors and mechanical components, and it is susceptible to damage when exposed to excessive heat, vibration, acceleration, or another type of stress. When any high value asset such as a computer or missile or sensitive piece of equipment is dropped or otherwise mishandled it poses a risk to the mission and end use of that system. For example, a missile may be carried on a ship. If the ship collides with another ship, the ship and everything onboard the ship may be subjected to certain levels of stress. For example, missiles and other equipment could potentially be tossed around or exposed to excessive vibration or acceleration, which may or may not damage the missiles. In the past, following a ship collision, the missiles onboard the colliding ships have needed to be shipped back to the manufacturer, at a great expense, to determine system health and efficacy. In these instances, the ship command had no way of knowing if the missiles remained in operational condition following the collision, which necessitated the missiles to be shipped to the manufacturer at a great expense of both money and time. The asset was out of service during this inspection period

In one respect, the monitoring device 116 can be affixed to missiles and other sensitive equipment (such as a bank of computers or a radar) and used to detect whether the missiles (or other equipment) have been exposed to excessive temperature or excessive levels of environmental or mechanical stress (e.g., when an emergency event occurs, such as a ship collision or a fire). For instance, when a missile is provided with the monitoring device 116, as is the case in the example of FIG. 1B, the monitoring device may record the temperature, acceleration, pressure (and/or any other parameter of the missile). If the ship carrying the missile collides with another missile, a user may use a scanning device to establish a connection with the monitoring device 116 and download the collected information, after which the collected information can be examined and used to determine whether the device or system that is monitored with the monitoring device 116 needs to be shipped back to the manufacturer for maintenance. In a simpler example, the long-term storage conditions of a pallet of ammunition, which is known to be sensitive to environmental conditions such as temperature or humidity can be known without destructive analysis of a sample specimen.

In another respect, the monitoring device 116 may use magnetic communications to connect with the outside world. An advantage of magnetic communications is that their emissions are difficult (if not impossible) to detect by the listening equipment of an adversary. However, magnetic communications have a range in the order of several feet, and for this reason, they require a scanning device to be brought in close proximity to the monitoring device 116 in order to download the information that is stored on the monitoring device 116. Other wired and wireless communication protocols are available.

In yet another respect, the monitoring device 116 may be used to provide a visual indicator of the conduction of sensitive equipment, which can be easily examined by personnel in real time to determine the combat worthiness of the sensitive equipment. As noted above, the monitoring device 116 may be disposed inside a container carrying sensitive equipment, and the notification device 106 may be disposed on the outside of the container. When the monitoring device 116 detects that the sensitive equipment has been subjected to excessive stress, the monitoring device 116 may communicate this information to the notification device 106, and the notification device 106 may display a visual indicator indicating that the sensitive equipment has been subjected to excessive stress. The visual indicator may then be noticed by personnel, and a decision can be made on how to proceed afterwards.

FIG. 1D shows a perspective view of the monitoring device 116, in accordance with one particular implementation. To illustrate the scale of the dimensions of the monitoring device 116, a quarter dollar coin 117 is placed over the top surface of the monitoring device 116. In some implementations, the enclosure of the monitoring device 116 may be formed of rubber, metal, and/or any other suitable manner. In one implementation, the monitoring device 116 may be affixed to the inner surface of a container by using double-sided adhesive tape and/or in any other suitable manner. As used throughout the disclosure, the term “container” may refer to any suitable type of receptacle that is used to carry a weapons system or other equipment. By way of example, a container may include a missile canister, a shipping crate, a missile launcher, a shipping container, or the enclosure of an electronics device (e.g., a computer rack, a computer case, an enclosure used to house radar equipment, etc.). FIG. 1E shows an example of a Landsmont box that could be used to hold the monitoring device 116. The Landsmont box includes a standardized hole pattern that allows it to be bolted to the exterior surface of shipping containers (or other types of containers) that are used by the military. The medical and aerospace industry also have similar containers and sensitive equipment requirements.

FIG. 1F is a diagram of an example of a system 100F, according to aspects of the disclosure. As illustrated, the system 100F may include the monitoring device 116, a scanning device 132, a data assessment system 134, and a customer device 136.

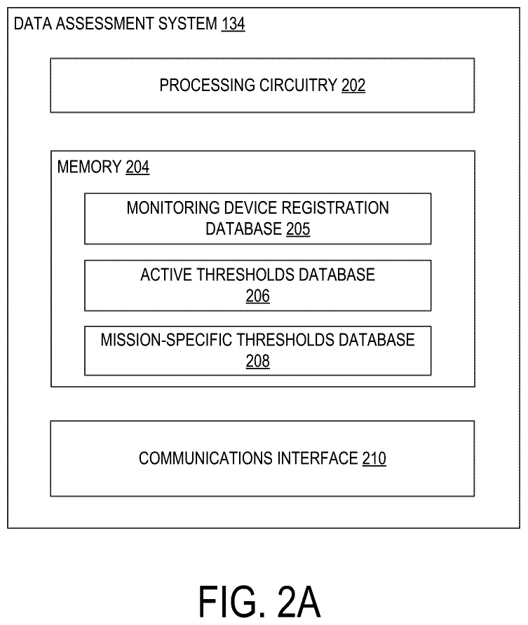

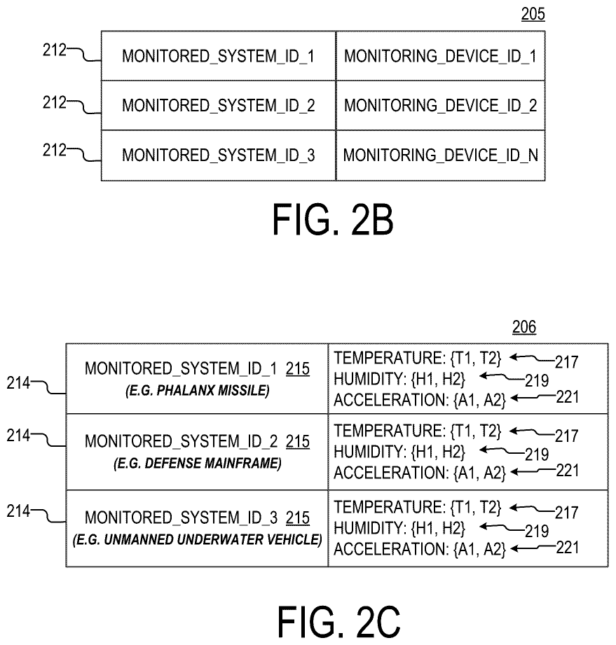

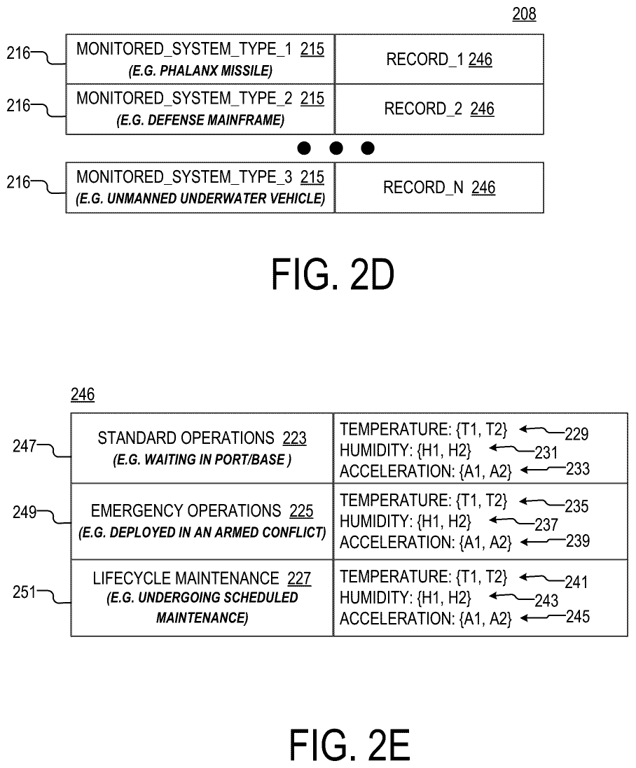

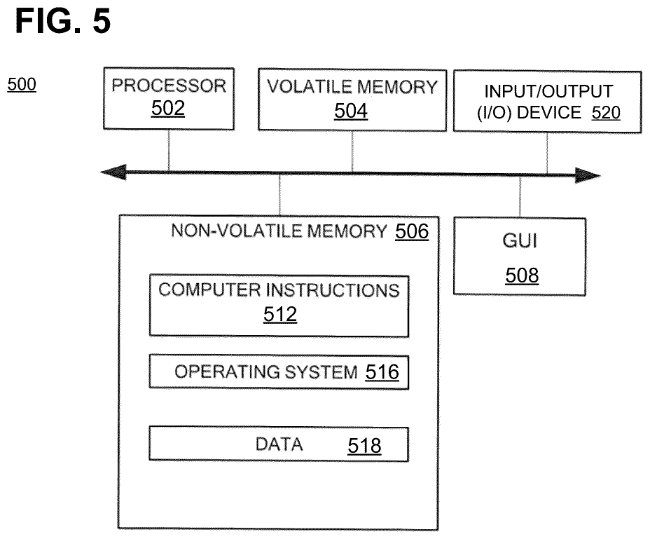

The scanning device 132 may include any suitable type of electronic device that is configured to receive data from the monitoring device 116 via a magnetic communication link 131. In some implementations, the scanning device may be a portable device, such as a smartphone and/or any other suitable type of electronic device that is provided with a magnetic communication interface. In some implementations, the scanning device 132 may have an architecture that is the same or similar to the architecture shown in FIG. 5. The data assessment system 134 may include any suitable type of distributed or integrated computing system. An example of one possible implementation of the data assessment system is provided further below with respect to FIG. 2A. The customer device 136 may include any suitable type of computing device. For example, the customer device 136 may include a laptop computer, a desktop computer, a smartphone, etc. In some implementations, the customer device 136 may have an architecture that is the same or similar to the architecture shown in FIG. 5.

In some implementations, the customer device 136 may be operated by the captain (or another officer) of a ship carrying a missile (or other equipment) that is monitored with the monitoring device 116. In some implementations, the scanning device 132 may be operated by enlisted personnel. In operation, the scanning device 132 may be arranged to establish a connection with the monitoring device 116 and download sensor measurements that are taken by the monitoring device 116. The connection may be established over a magnetic communication link 131. Next, the scanning device 132 may transmit the sensor measurements to the data assessment system 134. The transmission may be performed over a communications link 133. The communications link 133 may include any suitable type of wireless or wired communications link, such as an Internet Link, a local area network (LAN) link, a wide area network (WAN) link, and/or a WiFi or radio link. Next, the data assessment system 134 may compare each of the sensor measurements that are taken by the monitoring device 116 to one or more thresholds. When any of the thresholds is exceeded, the data assessment system 134 may transmit a notification to the customer device 136. Upon receiving the notification, the ship captain or another officer may take appropriate action, as the exceeding of the threshold may bear on the operational readiness of the equipment that is being monitored with the monitoring device 116. For example, if the notification indicates that the monitored equipment has been exposed to an excessive temperature or mechanical stress, the captain or other officer may order a further inspection of the equipment or order the equipment to be sent for maintenance.

FIG. 2A is a diagram of an example of the data assessment system 134, according to aspects of the disclosure. As illustrated, the data assessment system 134 may include a processing circuitry 202, a memory 204, and a communications interface 210. The processing circuitry 202 may include any of one or more general-purpose processors (e.g., x86 processors, etc.), one or more application-specific integrated circuits (ASICs), one or more Field-Programmable Gate Arrays (FPGAs), and/or any other suitable type of processing circuitry. The memory 204 may include any suitable type of volatile or non-volatile memory. For example, in some implementations, the memory 204 may include a hard disk, a solid-state drive, an NVRam drive, electrically-erasable programmable read-only memory (EEPROM), dynamic random-access memory (DRAM), and/or any other suitable type of memory. The communications interface 210 may include one or more of an 802.11 interface, an Ethernet interface, and/or any other suitable type of communications interface.

In some implementations, the memory 204 may be configured to store a registration database 205, an active thresholds database 206, and a mission-specific thresholds database 208. FIG. 2B shows an example of the registration database 205. The registration database 205 may be used to determine (or keep track of) which monitoring device is used to monitor what equipment. The registration database 205 may include a plurality of entries 212. Each entry may include an identifier of a respective monitoring device and an identifier of a monitored system, that is being monitored with the monitoring device. For example, if a monitoring device having the serial number ‘123’ is used to monitor a missile having the serial number ‘456’, the registration database 205 may contain an entry 212 that maps the two serial numbers to each other.

FIG. 2C is a diagram of the active thresholds database 206, according to aspects of the disclosure. The active thresholds database 206 may be used to map identifiers for different systems that are being monitored to corresponding values of thresholds for different environmental parameters. For example, if the monitoring device 116 is used to monitor the status of a Phalanx missile, the database 206 may identify a first temperature threshold T1 and a second temperature threshold T2, wherein T2<T1. Threshold T1 may be the maximum temperature that Phalanx missiles are generally capable of withstanding (before having to undergo maintenance or inspection), and threshold T2 may identify a temperature value that is above normal bounds for the Phalanx missile. As another example, the database 206 may identify humidity thresholds H1 and H2, where H2<H1. Threshold H1 may be the maximum humidity that Phalanx missiles are capable of withstanding (before having to undergo maintenance or inspection), and threshold H2 may identify a humidity value that is above normal bounds for the Phalanx missile. As yet another example, the database 206 may identify acceleration thresholds A1 and A2, where A2<A1. Threshold A1 may be the maximum acceleration that Phalanx missiles are capable of withstanding (before having to undergo maintenance or inspection), and threshold A2 may identify an acceleration value that is above normal bounds for the Phalanx missile. The acceleration measured may be an acceleration that is perpendicular to the sides of the missile, which would be experienced if the missile when the missile is tossed around in its container (rather than when the missile is flying).

As illustrated, the database 206 may include a plurality of entries 214. Each of the entries 214 may include an identifier 215 of a monitored system and threshold sets 217, 219, and 221. According to the present example, each of the threshold sets 217 is a set of temperature thresholds; each of the threshold sets 219 is a set of humidity thresholds; and each of the threshold sets 221 is a set of acceleration thresholds. In some implementations, the temperature threshold sets 217 in any two entries 214 may contain different threshold values. Additionally or alternatively, in some implementations, the humidity threshold sets 219 in any two entries 214 may contain different threshold values. Additionally or alternatively, in some implementations, the acceleration threshold sets 221 in any two entries 214 may contain different threshold values. Although in the example of FIG. 2C, each of the entries 214 contains thresholds for the same parameters, alternative implementations are possible in which any two of the entries 214 contain thresholds for different monitored parameters. For example, one entry 214 may contain a set 221 of acceleration thresholds, whereas another entry may lack a set of acceleration thresholds and have a set of pressure thresholds instead. Although in the example of FIG. 2C, each entry 214 includes thresholds for temperature, humidity, and acceleration, it will be understood that the present disclosure is not limited thereto. For example, each of the entries 214 may contain thresholds for only one parameter (e.g., temperature) or threshold sets for more than three parameters (e.g., temperature, humidity, acceleration, pressure, and vibration). Although in the example of FIG. 2C, each of the threshold sets 217, 219, and 221 includes two thresholds, alternative implementations are possible in which any of the threshold sets 217, 219, and 221 includes only one threshold value or more than two threshold values. In some implementations, the thresholds in each of the sets 217, 219, 221 may be based on operational specifications for each of the monitored systems that are provided by the systems' manufacturers. As can be readily appreciated, such specifications may identify the amounts of heat and mechanical stresses which the monitored systems can withstand. Although in the example of FIG. 2C, each of the entries 214 includes three threshold sets, alternative implementations are possible in which any of the entries 214 includes only one threshold set, or a different number of threshold sets such as two threshold sets or four threshold sets, etc.

FIG. 2D is a diagram of a mission-specific thresholds database 208. As illustrated, the database 208 may include a plurality of entries 216. Each of the entries 216 may map the identifier 215 for a different monitored system type to a corresponding record 246. The record 246 in each of the entries 216 may contain different information.

FIG. 2E is a diagram of a record 246, according to aspects of the disclosure. The record 246 may include entries 247, 249, and 251. Entry 247 may map a mission identifier 223 to threshold sets 229, 231, and 233. According to the present example, mission identifier 223 corresponds to a “standard operations” mission. Monitored equipment (e.g., a missile, etc.) may be on a “standard operations mission” when the monitored equipment (or a ship or vehicle carrying the monitored equipment) is waiting in port or at base or on routine operations. Threshold set 229 may identify one or more temperature thresholds that would be applied when the monitored equipment (or ship or vehicle carrying the monitored equipment) is on a standard operations mission. Threshold set 231 may identify one or more humidity thresholds that would be applied when the monitored equipment (or ship or vehicle carrying the monitored equipment) is on a standard operations mission. Threshold set 233 may identify one or more acceleration thresholds that would be applied when the monitored equipment (or ship or vehicle carrying the monitored equipment) is on a standard operations mission.

Entry 249 may map a mission identifier 225 to threshold sets 235, 237, and 239. According to the present example, mission identifier 225 corresponds to an “emergency operations mission”. Monitored equipment (e.g., a missile, etc.) may be on an “emergency operations mission” when the monitored equipment (or a ship or vehicle carrying the monitored equipment) is deployed in an armed conflict. Threshold set 235 may identify one or more temperature thresholds that would be applied when the monitored equipment (or ship or vehicle carrying the monitored equipment) is on an emergency mission. Threshold set 237 may identify one or more humidity thresholds that would be applied when the monitored equipment (or ship or vehicle carrying the monitored equipment) is on an emergency operations mission. Threshold set 239 may identify one or more acceleration thresholds that would be applied when the monitored equipment (or ship or vehicle carrying the monitored equipment) is on an emergency operations mission. Typically, the emergency mission would be represented by more open (broader) thresholds to allow for increased flexibility of system alarms and usage. Fewer alarm states would be represented to increase available assets in an emergency situation. Some commanders could change these threshold settings, and make them narrower to flag more threshold excursions and theoretically increase reliability of the assets being used in the emergency situation, but knowingly also reducing total inventory by use of narrower thresholds.

Entry 251 may map a mission identifier 227 to threshold sets 241, 243, and 245. According to the present example, mission identifier 227 corresponds to a “lifecycle maintenance” mission. Monitored equipment (e.g., a missile, etc.) may be on a “lifecycle maintenance” mission when the monitored equipment (or a ship or vehicle carrying the monitored equipment) is undergoing scheduled maintenance at the factory or a maintenance facility. Threshold set 241 may identify one or more temperature thresholds that would be applied when the monitored equipment (or ship or vehicle carrying the monitored equipment) is on a lifecycle maintenance mission. Threshold set 243 may identify one or more humidity thresholds that would be applied when the monitored equipment (or ship or vehicle carrying the monitored equipment) is on a lifecycle maintenance mission. Threshold set 245 may identify one or more acceleration thresholds that would be applied when the monitored equipment (or ship or vehicle carrying the monitored equipment) is on a lifecycle maintenance mission. The lifecycle maintenance mission may be the ideal time to remove suspect high value assets for further inspection, and may have the narrowest of the three threshold parameter settings.

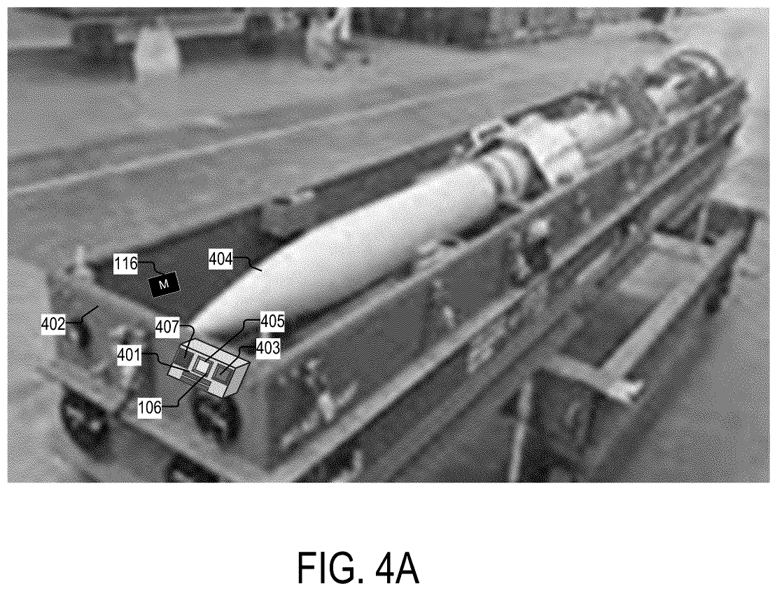

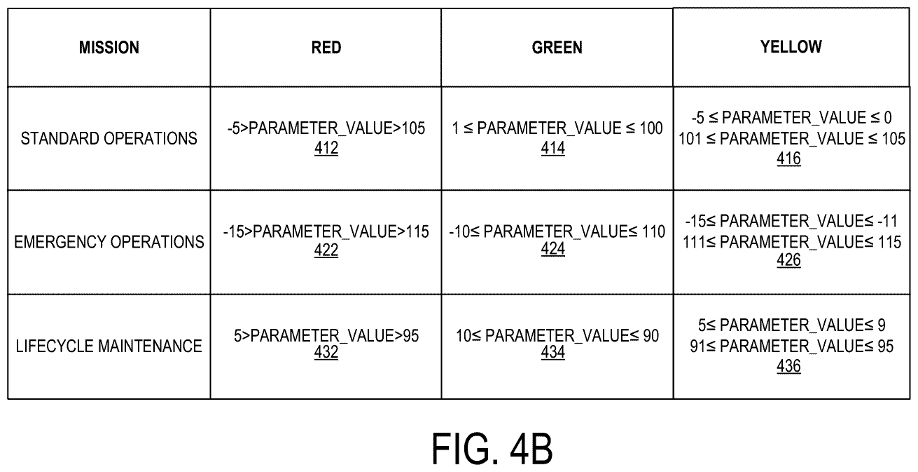

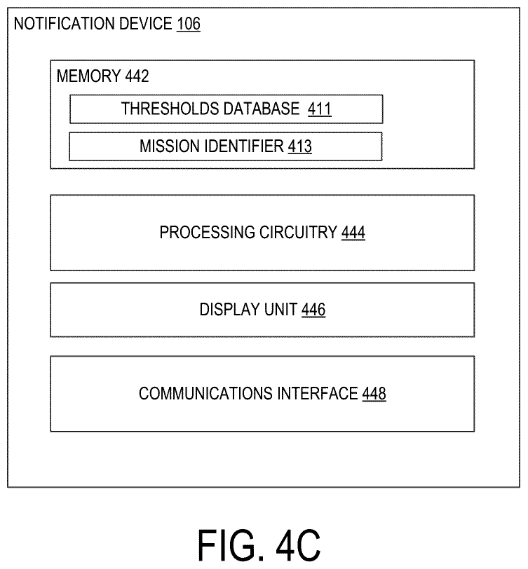

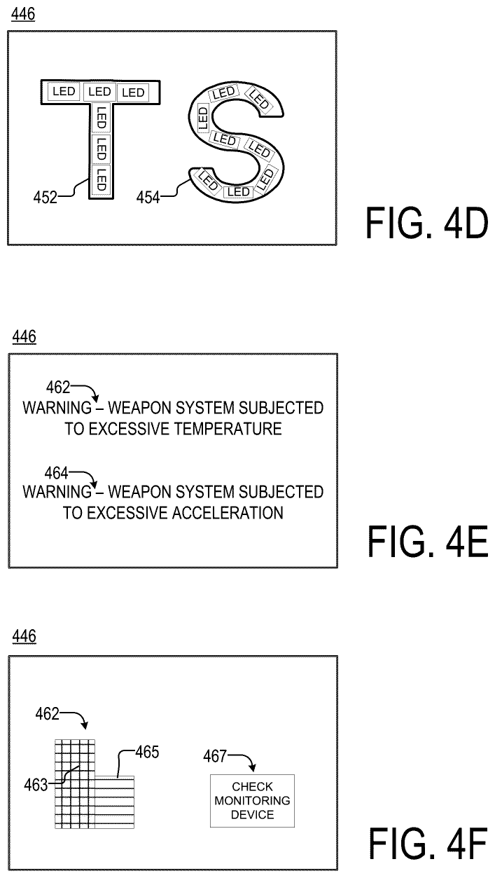

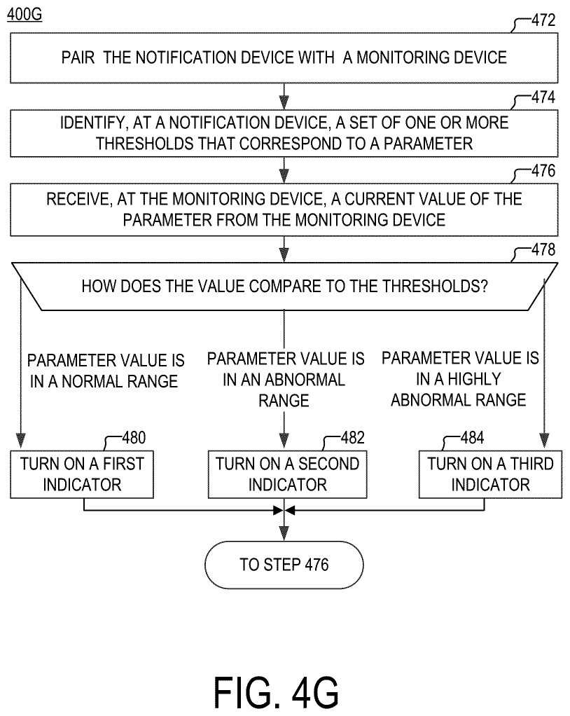

In some implementations, each of the entries 247, 249, and 251 may include a different set of temperature thresholds, a different set of humidity thresholds, and/or a different set of acceleration thresholds. In general, the thresholds in entry 251 may be lower than the thresholds in entries 247 and 249, and the thresholds in entry 247 may be lower than the thresholds in entry 249. In other words, under this arrangement, the thresholds associated with a “lifecycle maintenance” mission may be lower than the thresholds for “standard operations” and “emergency operations” missions, and thresholds for “standard operations” missions may be lower than the thresholds for “emergency operations” mission. An example of how the thresholds for a particular parameter (e.g., pressure, temperature, humidity, acceleration) can vary with the mission of a monitored system is discussed further below with respect to FIG. 4B.

Although in the example of FIG. 2E, each of the temperature, acceleration, and humidity threshold sets includes two thresholds, alternative implementations are possible in which any of the sets includes only one threshold. The system could have a manual (e.g., software driven) slider that changes thresholds in an infinite range as desired by an operator. Consider an example in which the sets 241 and 229 include one temperature threshold each. When a missile (or a combat platform on which the missile is based) is on a lifecycle maintenance mission, the temperature threshold in the set 241 may be compared against the current temperature of the missile, and a system condition alert may be issued when the threshold in the set 241 is crossed by the current temperature of the missile, indicating the asset came close to or exceeded a temperature of concern for the sensitive electronics within, and should be inspected further When the missile (or a combat platform on which the missile is based) is on standard operations mission, the temperature threshold in the set 229 may be compared against the current temperature of the missile, and a system condition alert may be issued when the threshold in set 229 is crossed by the current temperature of the missile. In this example, the temperature threshold in set 241 may be lower than the temperature threshold in the set 229. The net effect of making the threshold in set 241 lower would be that a smaller rise in the temperature of the missile would trigger the generation of a system condition alert when the missile (or carrier platform) is on a lifecycle maintenance mission (the easiest and most expedient time for a system to undergo inspection and maintenance) than when the missile is on a standard operations or emergency mission (the most difficult and least expedient time to do inspection or maintenance).

As used throughout the disclosure, the term database shall refer to one or more data structures and/or memory addresses/locations that are used to store information. For example, any of the databases discussed above with respect to FIGS. 2B-E may include a table (or a search tree, etc.) that is part of the software or firmware of the notification device 106 and/or the software of the data assessment system 134. Additionally or alternatively, in some implementations, any of the databases discussed above with respect to FIGS. 2B-E may be stored in the memory of the notification device 106, the data assessment system 134, and/or an external server. Stated succinctly, the present disclosure is not limited to any specific implementation of the databases that are discussed above with respect to FIGS. 2B-E.

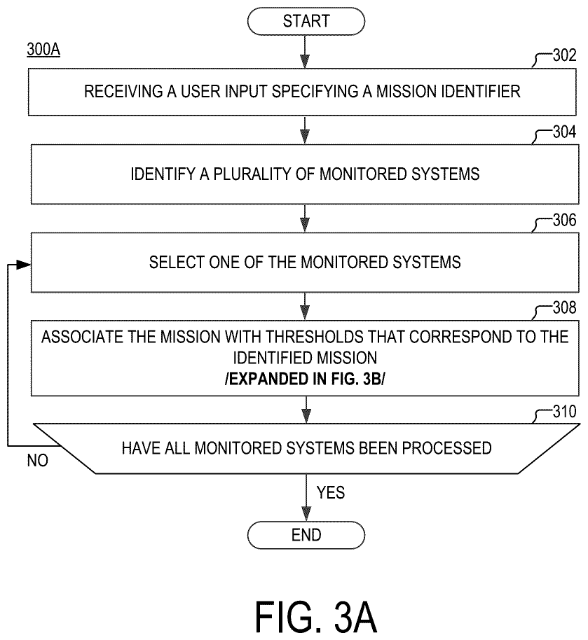

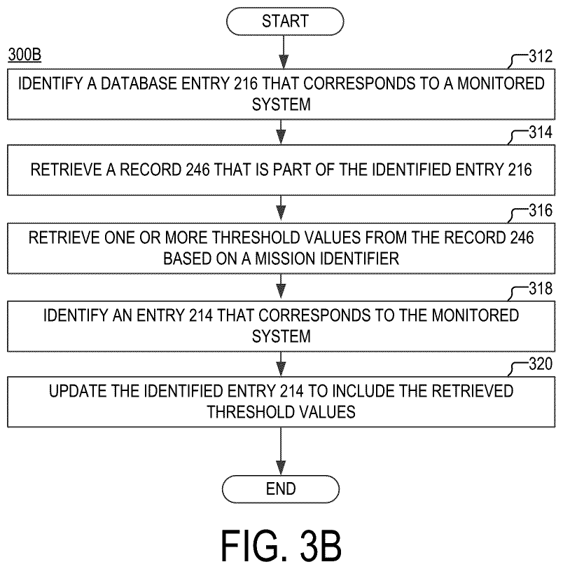

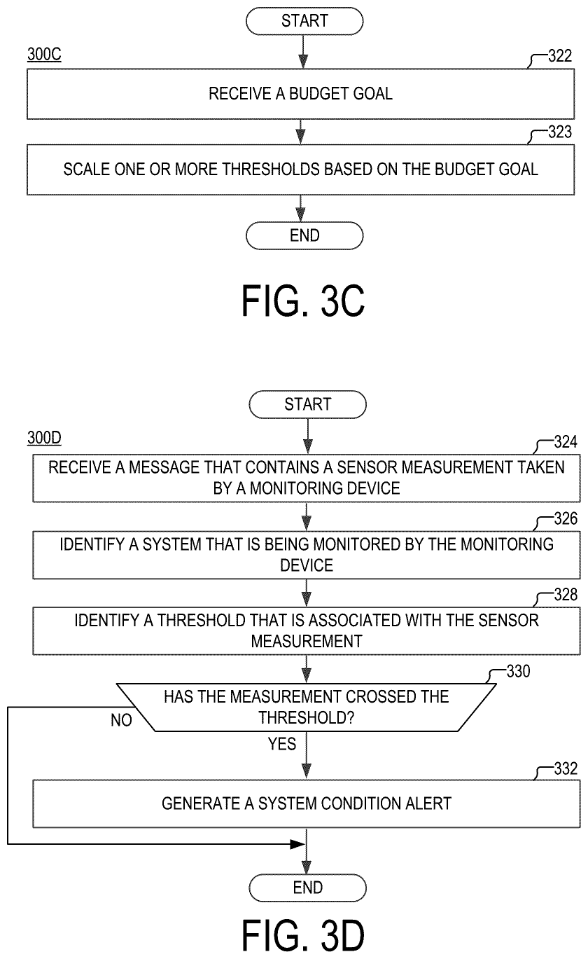

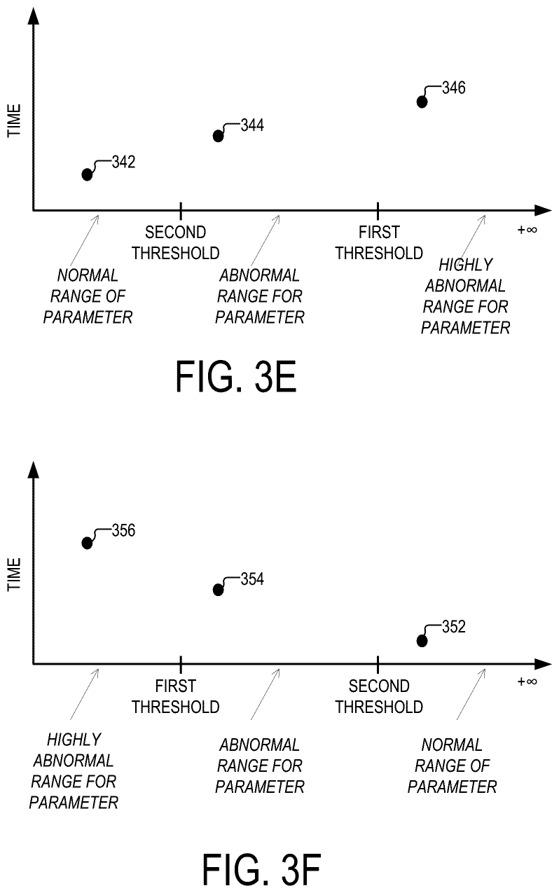

FIG. 3A is a flowchart of an example of a process 300A, according to aspects of the disclosure. According to the present example, the process 300A is performed by the data assessment system 134 (shown in FIG. 1F). However, the present disclosure is not limited to any specific entity or set of entities performing the process 300A. At step 302, the data assessment system 134 receives a user input specifying a mission identifier. At step 304, data assessment system 134 identifies a plurality of monitored systems. As noted above, a monitored system is any type of system or device that is being monitored with a monitoring device, such as the monitoring device 116 (shown in FIGS. 1C-D). Examples of monitoring systems include a missile (or another weapon or another system), a computer mainframe, radar equipment, etc. At step 306, the data assessment system 134 selects one of the plurality of monitored systems. At step 308, the data assessment system 134 associates the selected monitored system with one or more thresholds that correspond to the mission identifier (received at step 302). In some implementations, step 308 may be performed as discussed further below with respect to FIG. 3B. At step 310, the data assessment system 134 determines if all of the monitored systems (identified at step 304) have been processed. If all of the identified monitored systems have been processed, the process 300A ends. Otherwise, the process 300A returns to step 306 and another of the monitored systems (identified at step 304) is selected.

Although in the example of FIG. 3A, the mission identifier is received as user input, alternative implementations are possible in which the identifier is obtained from an artificial intelligence (AI) system. The system may include a neural network that is configured to classify a signature corresponding to the monitored equipment and/or a combat platform carrying the monitored equipment into a plurality of categories, where each category corresponds to a different mission identifier. The information may include location, speed, acceleration, news feeds, platform (ship, aircraft, battalion readiness level, human stress identifiers such as eye dilation or hormones), and/or any other suitable type of information. The AI would receive any number of mission identifiers (is the ship on high alert, is the operator stressed, have the world news outlets announced a major conflict or political or social unrest) and then automatically set the thresholds for system health alarm automatically. The monitoring system could also include automated escalation levels, where by the breath of the alarm notifications increases as the urgency of the situation or mission increases. When set in system maintenance mode the tolerances are typically narrower as discussed, and the notifications are only automatically sent to local mechanics or local logistical organizations. In an emergency war time situation the tolerances are typically broader as discussed, and the notifications are also broader to more and higher-level operators and organizations notifying them of asset availability automatically.

FIG. 3B is a flowchart of an example of a process 300B for associating a monitored system with one or more thresholds that correspond to a specific mission identifier, as specified by step 308 of the process 300A. At step 312, a database entry 216 is identified in the database 208 (shown in FIG. 2D) which corresponds to the monitored system. At step 314, a record 246 is retrieved from the identified database entry 216. At step 316, a search of the record 246 is performed to identify an entry that corresponds to the mission identifier. The identified entry may be the same or similar to any of the entries 247, 249, and 251, which are discussed above with respect to FIG. 2E. Afterwards, one or more thresholds that are part of the identified entry may be retrieved. At step 318, an entry 214 of the database 206 is identified that corresponds to the monitored system (see FIG. 2C). At step 320, the identified entry 214 is updated to include threshold values that are retrieved at step 316.

In one respect, the processes 300A and 300B enable the active thresholds for a particular monitored system to be set in accordance with the mission of the monitored system. For example, if a missile is on a standard operation mission, a temperature threshold for the missile may be set to a first value. On the other hand, if the missile is on an emergency operations mission, the threshold of the missile may be set to a first value that is higher than the first value. When the data assessment system 134 detects that the temperature threshold is exceeded, the data assessment system 134 may generate a system condition alert. In this regard, making the value of a temperature threshold mission-dependent would cause the data assessment system 134 to abstain from generating an alert while the missile is on an emergency operations mission, even though the system condition alert would be generated if the same temperature was detected while the missile is on a standard operations mission.

Although in the example of the processes 300A and 300B the active thresholds for a particular monitored system to are set based on a mission identifier, alternative implementations are possible in which the active threshold for a particular parameter is received as user input.

FIG. 3C is a flowchart of an example of a process 300C, according to aspects of the disclosure. According to the present example, the process 300C is performed by the data assessment system 134 (shown in FIG. 1F). However, the present disclosure is not limited to any specific entity or set of entities performing the process 300C. At step 322, the data assessment system 134 receives a budget goal. In some implementations, the budget goal may provide that a maintenance budget be increased or decreased from its current value. At step 323, the data assessment system 134 may scale all (or at least some) of the thresholds in the database 206 (shown in FIG. 2C).

As can be readily appreciated, when the budget goal specifies a reduction of the maintenance budget, at least some of the thresholds may be automatically scaled up, as permitted by the design specifications of the monitored systems to which the thresholds belong. Increasing the thresholds may cause a reduction in the rate at which system alerts are issued, which in turn could help decrease maintenance costs. Over time, this prognostic approach can be refined to match the statistical and operational observations of the asset performance and ultimately reduce total system lifecycle costs. In some implementations, the database 206 may be modified to include the maximum permitted threshold, for a particular environmental parameter, for each of the monitored systems that are listed in the database, and the data assessment system 134 may be arranged to increase any of the thresholds up to its maximum permitted value if necessitated by the budget goal.

In some implementations, the budget goal may be specific for a particular system condition. For example, the goal may specify a budget for the maintenance of a specific type of munition (e.g., a missile, a missile launcher, an artillery shell, a crate of bullet rounds, etc.) in the event of the munition being exposed to a high temperature. In such implementations, the data assessment system 134 may scale thresholds that correspond to the system condition (e.g., temperature thresholds). Furthermore, the data assessment system 134 may scale only temperature thresholds that are mapped to the specific type of munition (by the database 206).

FIG. 3D is a flowchart of an example of a process 300D, according to aspects of the disclosure. According to the present example, the process 300D is performed by the data assessment system 134 (shown in FIG. 1F). However, the present disclosure is not limited to any specific entity or set of entities performing the process 300D.

At step 324, the data assessment system 134 receives a message containing a measurement that is taken by a monitoring device. The monitoring device may be the same or similar to the monitoring device 116, which is discussed above with respect to FIGS. 1A-E. The message may be received from a scanning device, such as the scanning device 132. The message may contain an identifier of the monitoring device. The measurement may include a temperature measurement, a humidity measurement, an acceleration measurement, and/or any other suitable type of measurement.

At step 326, the data assessment system 134 performs a search of the registration database 205 (shown in FIG. 2B), to identify the type of system that is monitored with the monitoring device.

At step 328, the data assessment system 134 performs a search of the database 206, to identify a threshold that corresponds to the monitored system (identified at step 326). For example, if the measurement is a temperature measurement, the data assessment system 134 may identify a temperature threshold for the monitored system (identified at step 326). As another example, if the measurement is an acceleration measurement, the data assessment system 134 may identify an acceleration threshold for the monitored system (identified at step 326).

At step 330, the data assessment system 134 detects if the measurement has crossed the threshold. If the measurement has crossed the threshold, the process 300D proceeds to step 330. Otherwise, if the measurement is outside of the threshold, the process 300D proceeds to step 330.

At step 332, the data assessment system 134 generates a system condition alert and transmits the system alert to one or more recipients. In one aspect generating the system condition alert may include generating a message and transmitting the message. In some implementations, the message may include one or more of: (i) an identifier of the system that is being monitored (determined at step 326), (ii) an identifier of the monitoring device that has taken the measurement (received at step 324), (iii) the threshold (determined at step 326), (iv) an identifier of a current mission of the monitored system (or combat platform carrying the monitored system), (v) the value of the measurement that is received at step 324, and/or any other suitable information.

In some implementations, the message may be transmitted to a commanding officer (or another officer) of a combat platform (e.g., a ship, a truck, an aircraft, etc.) where the monitored system is located. Additionally or alternatively, in some implementations, the message may be transmitted to the address (e.g., an IP address, an email address, and/or any other type of address or identifier) of a specific computing or communications device that is located on the combat platform, such as a device that is known to be associated with a specific officer. In some implementations, the recipient of the system may be selected by performing a search of a directory that maps identifiers of different officers to identifiers of the systems (or munitions) which the officers oversee. Additionally or alternatively, in some implementations, the message may be transmitted to multiple recipients. As noted above, in some implementations, one or more of the recipients of the message may be selected based on the current mission of the monitoring device and/or the platform (or vehicle) that is carrying the monitoring device (from which the message is received at step 324). In such implementations, when the missions is a standard operations mission, only the officer that is directly in charge of a monitored system may be selected to receive the message. However, if the mission is an emergency operations mission, the officer immediately responsible as well as his or her supervisor may be contacted. In some implementations, one or more policy rules may be used to identify the message recipients. The policy rules may specify who should be provided with the message based on the current mission of the monitoring device and/or platform carrying the monitoring device.

Additionally or alternatively, in some implementations, the message may be transmitted to the system that is being monitored etc. Additionally or alternatively, in some implementations, the message may be transmitted to a built-in-test (BIT) subsystem of the monitored system. In such implementations, the BIT subsystem may use information in the message (along with information obtained from internal sensors) to refine its own assessment of the system that is being monitored and generate another alert if the criteria of the BIT subsystem for generating the alert are satisfied. In this way the BIT system (already inherent in most systems) and the external monitoring system work together for a more powerful and optimized total result.

In the example of FIG. 3D, the message (received at step 324) contains the value of only one measurement (e.g., the value of a temperature measurement, etc.). However, alternative implementations are possible, in which the message includes the values of multiple measurements (e.g., the value of a temperature measurement, the value of a humidity measurement, and the value of an acceleration measurement). In such implementations, each measurement may be compared to one or more thresholds that correspond to the measurement type (e.g., a temperature measurement may be compared to one more temperature thresholds, a humidity measurement may be compared to one or more humidity thresholds, and an acceleration measurement may be compared to one or more acceleration thresholds, etc.). When any of the thresholds is crossed, the data assessment system 134 may generate a system condition alert, such as the one discussed above with respect to step 332.

In some implementations, a monitoring device (such as the monitoring device 116) may be used to monitor an entire case of munitions. For example, the monitoring device 116 may be used to monitor the environment inside a launcher including multiple launch tubes, or inside a storage crate for missiles or artillery shells. In such implementations, the system condition alert may identify the case that is being monitored (e.g., the missile launcher or storage crate, etc.). Additionally or alternatively, in some implementations, the message may include an availability rating for the case. The availability rating may be a number, string, or alphanumerical string that identifies the percentage of munitions that are expected to function correctly in the event that they need to be used. In some implementations, the availability rating may be changed, by the data assessment system 134, when a message is received indicating that a parameter of the monitored system (e.g., temperature) has crossed a threshold. On the other hand, when a message is received indicating that the parameter is below the threshold, the availability rating may be left unchanged. In some implementations, the availability rating may be used by a ship captain, battalion general or another officer to judge the battle-readiness of an entire array of munitions. The same information may be of interest to logistical and acquisition personnel for planning purposes.

FIG. 3E shows a plot of 300E of possible values of a parameter that is being measured by a monitoring device, such as the monitoring device 116. The parameter may include temperature, humidity, acceleration, and/or any other suitable type of parameter. The parameter may have a normal range, an abnormal range, and a highly abnormal range. The ranges are defined by a first threshold and a second threshold. The ranges may be specific to a particular system that is being monitored with the monitoring device, such as the missile 112 (shown in FIG. 1B). For example, when the parameter is temperature, the normal operating range of the parameter, may be the normal temperature range for the monitored system. The abnormal range may be a range in which the temperature of the monitored system would be considered high, but unlikely to result in damage to the monitored system. The highly abnormal range may be a range in which the monitored system is at risk of sustaining damage and can optionally be further classified into long-term or short-term damage. Also shown in FIG. 3E are values 342, 344, and 346 for the parameter. The value 342 is in the normal operating range of the monitored parameter, the value 344 is in the abnormal range for the parameter, and the value 346 is in the highly abnormal range for the parameter. Under the nomenclature of the present disclosure, value 342 is considered to be outside of the first threshold and the second threshold, value 344 is considered to have crossed the second threshold, while remaining outside of the first threshold, and value 346 is considered to have crossed both the first threshold and the second threshold.

FIG. 3F shows a plot of 300F of possible values of a parameter that is being measured by a monitoring device, such as the monitoring device 116. The parameter may include temperature, humidity, acceleration, and/or any other suitable type of parameter. The parameter may have a normal range, an abnormal range, and a highly abnormal range. The ranges are defined by a first threshold and a second threshold. Also shown in FIG. 3E are values 352, 354, and 356 for the parameter. The value 352 is in the normal operating range of the monitored parameter, the value 354 is in the abnormal range for the parameter, and the value 346 is in the highly abnormal range for the parameter. Under the nomenclature of the present disclosure, value 352 is considered to be outside of the first threshold and the second threshold, value 354 is considered to have crossed the second threshold, while remaining outside of the first threshold, and value 356 is considered to have crossed both the first threshold and the second threshold.

FIG. 4A is a diagram illustrating an example of another use case for the monitoring device 116 and the notification device 106. Shown in FIG. 4A is a shipping crate 402 for a missile 404. Inside the shipping crate 402, the monitoring device 116 is placed. On the outside surface of the shipping crate 402, a monitoring device 116 is mounted. The monitoring device 116 includes a display unit 401. The display unit 401, includes indicators 403, 405, and 407. Indicator 403 may include a green light-emitting diode (LED), indicator 405 may include a yellow LED, and indicator 407 may include a red LED. In operation, the monitoring device 116 may receive temperature measurements (or measurements of another parameter) that are taken by the monitoring device, and process each of the measurements. For example, when a measurement is in the normal operating range for the parameter, the notification device 106 may turn on the green light (i.e., turn on indicator 403) while keeping the red and yellow lights turned off; when the measurement is in the abnormal range for the parameter, the notification device may turn on the yellow light, while keeping the green and red lights off; and when the measurement is in the highly abnormal range for the parameter, the notification may turn on the red light while keeping the yellow and green lights off. These light configurations are especially useful for the on-sight, real time reading and analysis of system health in urgent situations, where electronic reading or monitoring is precluded due to time, urgency or other considerations. For example, these light configurations could be easily read by an untrained personnel at a glance in a fire or other catastrophe to provide leadership with basic asset availability information.

In one respect, FIG. 4A illustrates a scenario in which the notification device 106 is mounted on an exterior surface of a container carrying sensitive equipment. The mounting of the notification allows the display unit 401 of the notification device to be easily observed by enlisted personnel. The color-encoding of the display unit 401 further permits enlisted personnel to easily make judgments as to the amount of stress, which is experienced by sensitive equipment being monitored (e.g., the missile 404 in the example of FIG. 4A). For example, if the shipping crate 402 is aboard a ship, and the ship experiences a collision, the notification devices would permit the enlisted personnel whether missile 404 is at risk of having been damaged as a result of the collision. In some implementations, the notification device 106 may be provided with a bracket for mounting the notification device 106 on the surface of the shipping crate 402.

FIG. 4B is a diagram of different threshold sets, which can be used by the monitoring device 116 to determine whether to turn on any of the indicators 403, 405, and 407. The thresholds in FIG. 4B are meant to be compared against the value of a parameter (e.g., temperature, acceleration, etc.) that is measured by the monitoring device 116 and provided to the notification device 106. The thresholds shown in FIG. 4B are used to form respective conditions 412-436. FIG. 4B illustrates that the green light (or the red and yellow lights) can be turned on under different circumstances, depending on the mission on which the monitored system (e.g., the missile 404) or a carrier of the monitored system (e.g., a ship or a truck) is deployed. For example, if the monitored system (or carrier platform) is on a standard operations mission: (i) the notification device 106 may turn on the red light when condition 412 is satisfied by the value of the parameter that is measured by the monitoring device 116, (ii) the notification device 106 may turn on the green light when condition 414 is satisfied by the value of the parameter that is measured by the monitoring device 116, and (iii) the notification device 106 may turn on the yellow light when condition 416 is satisfied by the value of the parameter that is measured by the monitoring device 116. As another example, if the monitored system (or carrier platform) is on an emergency operations mission: (i) the notification device 106 may turn on the red light when condition 422 is satisfied by the value of the parameter that is measured by the monitoring device 116, (ii) the notification device 106 may turn on the green light when condition 424 is satisfied by the value of the parameter that is measured by the monitoring device 116, and (iii) the notification device 106 may turn on the yellow light when condition 426 is satisfied by the value of the parameter that is measured by the monitoring device 116. As yet another example, if the monitored system (or carrier platform) is on a lifecycle maintenance mission: (i) the notification device 106 may turn on the red light when condition 432 is satisfied by the value of the parameter that is measured by the monitoring device 116, (ii) the notification device 106 may turn on the green light when condition 434 is satisfied by the value of the parameter that is measured by the monitoring device 116, and (iii) the notification device 106 may turn on the yellow light when condition 436 is satisfied by the value of the parameter that is measured by the monitoring device 116. The above examples assume that only one light in the display unit 446 may be turned on at a time—that is, when the green light is turned on, the red and yellow lights are turned off, similarly when the yellow light is turned on, the green and red lights are turned off, and when the red light is turned on, the green and yellow lights are turned off.

FIG. 4C is a diagram of the notification device 106, in accordance with one particular implementation. As illustrated, the notification device 106 may include a memory 442, a processing circuitry 444, a display unit 446, and a communications interface 448. The memory 442 may include any suitable type of volatile or non-volatile memory. In some implementations, the memory 442 may include a hard disk, a solid-state drive, an NVRam drive, electrically-erasable programmable read-only memory (EEPROM), dynamic random-access memory (DRAM), and/or any other suitable type of memory. The processing circuitry 444 may include any of: one or more general-purpose processors (e.g., x86 processors, etc.), one or more application-specific integrated circuits (ASICs), one or more Field-Programmable Gate Arrays (FPGAs), and/or any other suitable type of processing circuitry. The communications interface 448 may include one or more of an 802.11 wireless interface, an Ethernet interface, and/or any other suitable type of communications interface. The memory 442 may be configured to store a thresholds database 411 and a mission identifier 413. The thresholds database 411 may identify different sets of thresholds for a parameter that is being measured by a monitoring device, as well as the type of mission which each of the sets is associated with. In some implementations, the thresholds database 411 may contain the same information as the table shown in FIG. 4B. Additionally or alternatively, in some implementations, the thresholds database 411 may contain the same information as any of the records 246, which are discussed above with respect to FIGS. 2D-E. Additionally or alternatively, in some implementations, the thresholds database 411 may only store the thresholds that correspond to the current mission of the system that is being monitored (or a carrier platform). The mission identifier 413 may identify the current mission of the monitored system (or carrier platform). In some implementations, the mission identifier 413 may be used to key into the thresholds database 411 to retrieve the threshold values that need to be compared against a measurement that is provided by the monitoring device 116. In some implementations, the mission identifier may be received as user input (e.g., via the communications interface 448, etc.). Additionally or alternatively, the values of different thresholds that are stored in the thresholds database 411 may be received as user input (e.g., via the communications interface 448).

According to the present example, the notification device 106 is an integrated system. Specifically, in the present example, the memory 442, processing circuitry 444, display unit 446, and communications interface 448 are housed in the same housing enclosure (not shown). However, alternative implementations are possible in which the notification device 106 is implemented as a distributed system. In such implementations, the processing circuitry 444 may be disposed in one housing enclosure and the display unit 446 may be disposed in a different housing enclosure. The display unit 446 may be connected to the processing circuitry 444 via a wired or wireless channel. For example, in some implementations, the display unit 446 may use a Bluetooth connection to receive, from the processing circuitry 444, data that is desired to be displayed from the processing circuitry 444.

According to the example of FIGS. 1A-4A, the notification device 106 and the monitoring device 116 are separate from each other. However, alternative implementations are possible in which the notification device 106 and the monitoring device 116 are integrated together. In such implementations, the monitoring device 116 may be provided with a display unit, such as the display unit 446, and configured to perform any of the operations that are described as being performed by the notification device 106.

FIG. 4D is a diagram of the display unit 446 in accordance with one particular implementation. In the example of FIG. 4D, the display unit includes an LED array 452 and an LED array 454. As is discussed further below, LED arrays 452 and 454 are an example of one type of indicator. In some implementations, the display unit 446 may include only one of LED arrays 452 and 454 and in other examples, the display unit 446 may include more than two LED arrays.

LED array 452 may be driven by temperature measurements that are provided to the notification device 106 by the monitoring device 116. LED array 452 may include a plurality of LEDs that are arranged to define the shape of the letter ‘T’. When temperature values reported by the monitoring device 116 are in a normal operating range, the notification device 106 may cause the LEDs in the LED array 452 to emit a green light. When the temperature values reported by the monitoring device 116 are in an abnormal range, the notification device 106 may cause the LEDs in the LED array 452 to emit a yellow light. And when the temperature values reported by the monitoring device 116 are in a highly abnormal range, the notification device 106 may cause the LEDs in the LED array 452 to emit a red light.

LED array 454 may be driven by acceleration measurements that are provided to the notification device 106 by the monitoring device 116. LED array 454 may include a plurality of LEDs that are arranged to define the shape of the letter ‘S’—where ‘s’ stands for “mechanical stress”. When acceleration values reported by the monitoring device 116 are in a normal operating range, the notification device 106 may cause the LEDs in the LED array 454 to emit a green light. When the acceleration values reported by the monitoring device 116 are in an abnormal range, the notification device 106 may cause the LEDs in the LED array 454 to emit a yellow light. And when the acceleration values reported by the monitoring device 116 are in a highly abnormal range, the notification device 106 may cause the LEDs in the LED array 454 to emit a red light.

FIG. 4E is a diagram of the display unit 446 in accordance with one particular implementations. In the example of FIG. 4E, the display unit includes a general-purpose display panel, such as a liquid crystal display (LCD) or an LED panel. The display panel may be the same or similar to display panels that are found in smart phones or other similar devices. In the example of FIG. 4E, the display unit may display indicators 462 and 464.

Indicator 462 may be driven by temperature measurements that are provided to the notification device 106 by the monitoring device 116. For example, when the temperature measurements are within the normal operating range for the temperature, the indicator 462 may include a first text message or a first graphical image. When the temperature measurements are within an abnormal range, the indicator 462 may include a second text message or a second graphical image. And when the temperature measurements are within a highly abnormal range, the indicator 462 may include a third text message or a third graphical image.

Indicator 464 may be driven by acceleration measurements that are provided to the notification device 106 by the monitoring device 116. For example, when the acceleration measurements are within the normal operating range for the acceleration, the indicator 464 may include a first text message or a first graphical image. When the acceleration measurements are within an abnormal range, the indicator 464 may include a second text message or a second graphical image. And when the acceleration measurements are within a highly abnormal range, the indicator 464 may include a third text message or a third graphical image.

FIG. 4F shows another possible implementation of the indicator 462. In this implementation, the indicator 462 includes a bar 463 which indicates the value of the parameter that is being measured, and a bar 465 which indicates the value of the threshold against which the parameter value is compared. In some respects, bars 463 and 465 may provide an indication of the extent by which the threshold is exceeded (or not exceeded). Also shown in FIG. 4F is an indicator 467, which is displayed when the monitoring device 116 experiences a status change. The status change may be a low-battery condition, an internal failure of the monitoring device 116 and/or any other suitable type of status change. When the monitoring device 116 status change, the monitoring device 116 may transmit to the notification device 106 a message indicating the status change, and the notification device may display the indicator 467 in response to the message. The indicator 467 may contain an indication of the type of the status change (e.g., “low battery” or “insufficient memory”) and/or any other suitable information.

FIG. 4E is provided as an example only, in some implementations, indicator 462 may be configured to provide one or more of: (i) an identifier of a type of parameter (e.g., humidity, temperature, acceleration, etc.), (ii) the value of the parameter (e.g., 79C or 95 m/s², etc.), (iii) the value of the threshold, (iv) an indication of the threshold is exceeded or not, and (v) an indication of the current mission of the monitored system (or combat platform on which the monitored system is based), etc. In some implementations, the indication of whether threshold is exceeded by a particular measurement may be color-coded. For example, the notification device 106 may display the string “79C” in red, green, or yellow depending on whether thresholds T1 and T2 (T2<T1) are exceeded. If 79<T2<T1, the string “79C” may be displayed in green. If T2<79<T1, the string “79C” may be displayed in yellow. If T2<T1<79, the string ‘79C’ may be displayed in red.

In the example of FIG. 4E, the display unit 446 is a graphic display device, such as an LED display that is customarily found in smartphones, and the indicators 462 and 464 are graphic indicators. However, alternative implementations are possible in which the display unit 446 includes a speaker. In such implementations, each of indicators 462 and 464 may include a set of audible tones. For example, indicator 462 may include a first tone that is played when a temperature measurement does not exceed either of thresholds T1 and T2, a second tone that is played when the temperature measurement exceeds threshold T1 only, and a third tone that is played when the temperature measurement exceeds both thresholds T1 and T2. Throughout the disclosure, and as permitted by context, the terms “display unit” and “output unit” are used interchangeably. In some implementations, the display unit 446 may be replaced (or supplemented) with an audio speaker.

Furthermore, it will be understood that alternative implementations are possible in which the display unit 446 is implemented as a tactile feedback device. In such implementations, indicator 462 may be implemented as a first tactile pin that is receded when the value of a first parameter (e.g., temperature) is below a first threshold, and extended when the value of the first parameter has exceeded the first threshold. In some implementations, the first pin may “pop up” after the first parameter has crossed the first threshold and remain this way until it is reset (e.g., pushed back) by an operator. Similarly, indicator 464 may be implemented as a second tactile pin that is receded when the value of a second parameter (e.g., acceleration) is below a second threshold, and extended when the value of the second parameter has crossed the second threshold. In some implementations, the second pin may “pop up” and remain extended after the second parameter has crossed the second threshold until it is reset (e.g., pushed back) by an operator. In some implementations, the second pin may “pop up” after the first parameter has crossed the first threshold and remain this way until it is reset (e.g., pushed back) by an operator.

FIG. 4G is a flowchart of an example of a process 400G, according to aspects of the disclosure. According to the present example, the process 400G is performed by the monitoring device 116. However, alternative implementations are possible in which the process 400G is performed by another device or group of devices.

At step 472, the notification device 106 and the monitoring device 116 are paired. Pairing the notification device 106 and the monitoring device 116 may include at least one of (i) establishing a connection between the notification device and the monitoring device 116, (ii) receiving at the notification device 106 (from the monitoring device 116) an identifier corresponding to the system (e.g., missile, etc.) that is being monitored by the monitoring device 116, (iii) receiving an identifier corresponding to the monitoring device 116. The identifier corresponding to the monitoring device 116 may be an identifier that is subsequently used to transmit and receive messages from the monitoring device 116. The identifier may include an address or another unique identifier of the monitoring device or an identifier of a particular frequency or a communications channel that is used by the monitoring device to transmit data. According to the present example, the connection is established by using a magnetic communications channel. However, the disclosure is not limited to using any specific communications technology for establishing the connection.

Although in the present example, the identifier of the monitored system and the identifier corresponding to the monitored device are received from the notification device 116, alternative implementations are possible in which the identifiers are received from the scanning device 132. In such implementations, the scanning device may obtain the identifiers (e.g., by scanning bar codes on the monitored system or monitoring device 116, or as a result of receiving user input).

At step 474, the notification device 106 identifies a set of one or more thresholds that correspond to a parameter and stores the received thresholds in the memory 442. The parameter may include temperature, acceleration, humidity, pressure, and/or any other suitable type of parameter that is measured by the monitoring device 116. According to the present example, the set of thresholds includes a first threshold and a second threshold. Together, the first threshold and the second threshold may define a normal operating range for the parameter, an abnormal range of the parameter, and a highly abnormal range for the parameter. According to the present example, the set of thresholds is received as user input (e.g., via the communications interface 448). For example, in some implementations, set of thresholds may input by the user into the scanning device 132 (shown in FIG. 1F), and transmitted to the notification device 106 via a wireless connection that is established between the notification device 106 and the scanning device 132.

Although in the present example, the parameter thresholds are received as user input, alternative implementations are possible in which the one or more parameter thresholds are identified based on a mission identifier that is received the notification device 106. The mission identifier may be received as user input via the scanning device 132 or from the system 134. In such implementations, the notification device 132 may perform a search of the database 411 based on the mission identifier and retrieve the one or more thresholds as a result of the search. Additionally or alternatively, in some implementations, the database 411 may be the same or similar to the database 206. In such implementations, the notification device 106 may use an identifier of a monitored system (received at step 472) to perform a search of the database 411 and retrieve the one or more thresholds that correspond to the monitored system as a result of the search.

At step 476, the notification device 106 receives, from the monitoring device 116, a value of the parameter that is measured by the monitoring device 116. For example, if the parameter is temperature, the value may be a temperature measurement that is taken by the monitoring device 116. As another example, if the parameter is acceleration, the value may be an acceleration measurement that is taken by the monitoring device 116, etc. In some implementations, the notification device 106 may receive, at step 476, a message that includes the parameter value as well as a parameter identifier, which is identifies the type of parameter whose value is provided in the message.

At step 478, the notification device 106 compares the parameter value (received at step 476) to the values of the first and second thresholds (received at step 472). If the parameter value has failed to cross both the first threshold and the second threshold (and/or if the value is in a normal range for the parameter), the process 400G proceeds to step 480. If the parameter value has crossed the second threshold, while remaining outside of the first threshold (and/or if the value is in an abnormal range for the parameter), the process 400G proceeds to step 482. If the parameter value has crossed both the first threshold and the second threshold (and/or if the value is in a highly abnormal range for the parameter), the process 400G proceeds to step 482.

At step 480, the notification device 106 turns on a first indicator. In one example, turning on the first indicator may include turning on a green LED (e.g., see indicator 403, shown n FIG. 4A). In another example, turning on the first indicator may include causing an LED array to emit green light (e.g., see LED arrays 452 and 454, shown in FIG. 4D). In yet another example, turning on the first indicator may include displaying a text message or an image that is indicative that the parameter is in its normal range.

At step 482, the notification device 106 turns on a second indicator. In one example, turning on the second indicator may include turning on a yellow LED (e.g., see indicator 405, shown n FIG. 4A). In another example, turning on the second indicator may include causing an LED array to emit yellow light (e.g., see LED arrays 452 and 454, shown in FIG. 4D). In yet another example, turning on the second indicator may include displaying a text message or an image that is indicative that the parameter is in a highly abnormal range.

At step 484, the notification device 106 turns on a third indicator. In one example, turning on the third indicator may include turning on a red LED (e.g., see indicator 407, shown n FIG. 4A). In another example, turning on the third indicator may include causing an LED array to emit red light (e.g., see LED arrays 452 and 454, shown in FIG. 4D). In yet another example, turning on the third indicator may include displaying a text message or an image that is indicative that the parameter is in an abnormal range.

Referring to FIG. 5, computing device 500 that can be used to implement, at least in part, the computing system 300. As illustrated, the computing device 500 may include processor 502, volatile memory 504 (e.g., RAM), non-volatile memory 506 (e.g., a hard disk drive, a solid-state drive such as a flash drive, a hybrid magnetic and solid-state drive, etc.), graphical user interface (GUI) 508 (e.g., a touchscreen, a display, and so forth) and input/output (I/O) device 520 (e.g., a mouse, a keyboard, etc.). Non-volatile memory 506 stores computer instructions 512, an operating system 516 and data 518 such that, for example, the computer instructions 512 are executed by the processor 502 out of volatile memory 504. Program code may be applied to data entered using an input device of GUI 508 or received from I/O device 520.

Processor 502 may be implemented by one or more programmable processors executing one or more computer programs to perform the functions of the system. As used herein, the term “processor” describes an electronic circuit that performs a function, an operation, or a sequence of operations. The function, operation, or sequence of operations may be hard-coded into the electronic circuit or soft coded by way of instructions held in a memory device. A “processor” may perform the function, operation, or sequence of operations using digital values or using analog signals. In some embodiments, the “processor” can be embodied in an application-specific integrated circuit (ASIC). In some embodiments, the “processor” may be embodied in a microprocessor with associated program memory. In some embodiments, the “processor” may be embodied in a discrete electronic circuit. The “processor” may be analog, digital or mixed-signal. In some embodiments, the “processor” may be one or more physical processors or one or more “virtual” (e.g., remotely located or “cloud”) processors.

The processes described herein are not limited to use with hardware and software of FIG. 1; they may find applicability in any computing or processing environment and with any type of machine or set of machines that is capable of running a computer program. The processes described herein may be implemented in hardware, software, or a combination of the two. The processes described herein may be implemented in computer programs executed on programmable computers/machines that each includes a processor, a non-transitory machine-readable medium or another article of manufacture that is readable by the processor (including volatile and non-volatile memory and/or storage elements), at least one input device, and one or more output devices. Program code may be applied to data entered using an input device to perform any of the processes described herein and to generate output information.

The system may be implemented, at least in part, via a computer program product, (e.g., in a non-transitory machine-readable storage medium such as, for example, a non-transitory computer-readable medium), for execution by, or to control the operation of, data processing apparatus (e.g., a programmable processor, a computer, or multiple computers). Each such program may be implemented in a high-level procedural or object-oriented programming language to work with the rest of the computer-based system. However, the programs may be implemented in assembly, machine language, or Hardware Description Language. The language may be a compiled or an interpreted language, and it may be deployed in any form, including as a stand-alone program or as a module, component, subroutine, or another unit suitable for use in a computing environment. A computer program may be deployed to be executed on one computer or multiple computers at one site or distributed across multiple sites and interconnected by a communication network. A computer program may be stored on a non-transitory machine-readable medium that is readable by a general or special purpose programmable computer for configuring and operating the computer when the non-transitory machine-readable medium is read by the computer to perform the processes described herein. For example, the processes described herein may also be implemented as a non-transitory machine-readable storage medium, configured with a computer program, where upon execution, instructions in the computer program cause the computer to operate in accordance with the processes. A non-transitory machine-readable medium may include but is not limited to a hard drive, compact disc, flash memory, non-volatile memory, volatile memory, magnetic diskette and so forth but does not include a transitory signal per se.

Having described preferred embodiments, which serve to illustrate various concepts, structures and techniques, which are the subject of this patent, it will now become apparent that other embodiments incorporating these concepts, structures and techniques may be used. Accordingly, it is submitted that the scope of the patent should not be limited to the described embodiments but rather should be limited only by the spirit and scope of the following claims.

1. A method for use in a computing device, the method comprising:
receiving a first value of a first parameter, the first value being generated by a monitoring device that is arranged to monitor a respective monitored system;
retrieving, from a memory, a first threshold that is associated with the first parameter and the respective monitored system; and
generating a first system condition alert when the first value has crossed the first threshold.

2. The method of claim 1, further comprising receiving a user input specifying the first threshold, and storing the first threshold in the memory, wherein the generating the first system condition alert includes: identifying a current mission requirement of the monitoring device or a platform carrying the monitoring device, identifying one or more recipients based on the current mission requirement, and transmitting the first system condition alert to the identified recipients.

3. The method of claim 1, wherein the first system condition alert includes at least one of an identifier of a current mission requirement, the first value of the first parameter, and the first threshold.

4. The method of claim 1, wherein the first threshold is retrieved from a database that stores a plurality of thresholds for the first parameter, each of the plurality of thresholds being associated with a different type of monitored system, the method further comprising receiving user input specifying a budget for a system condition of one of monitored system types, and scaling the plurality of thresholds in response to the user input.

5. The method of claim 1, wherein the first parameter includes at least one of vibration, acceleration, humidity, pressure, and temperature, and the computing device includes one of a scanning device and/or a data assessment system.

6. The method of claim 1, wherein the respective monitored system includes one of a missile, an aircraft, a watercraft, a radar, or a combat electronics system.

7. The method of claim 1, wherein the respective monitored system is based on a combat platform, the method further comprising, receiving a user input identifying a mission for the combat platform, selecting a threshold value for the first threshold based on the mission, and setting the first threshold to equal the threshold value.

8. The method of claim 7, wherein the mission for the combat platform is selected from a group including a standard operations mission, an emergency operations mission, and a lifecycle maintenance mission, and selecting the threshold value for the first threshold, includes performing a search of a database based on an identifier of the mission to identify a threshold value that is mapped by the database to the identifier of the mission.

9. The method of claim 7, further comprising selecting a recipient for the first system condition alert based on the mission for the combat platform, wherein generating the first system condition alert includes transmitting the first system condition alert to the selected recipient.

10. The method of claim 1, wherein the respective monitored system includes a munition that is part of a plurality of munitions that are stored in a same case or pallet, the method further comprising changing an availability rating for the plurality of munitions when the first value has crossed the first threshold, and leaving the availability rating unchanged when the first value has not crossed the first threshold.

11. The method of claim 1, further comprising:
receiving a second value of a second parameter, the second value being received at the computing device from the monitoring device;
retrieving a second threshold that is associated with the second parameter and the respective monitored system; and
generating a second system condition alert when the second value has crossed the second threshold.

12. A system comprising:
a memory; and
at least one processor operatively coupled to the memory, the at least one processor being configured to perform the operations of:
receiving a first value of a first parameter, the first value being received from a monitoring device that is arranged to monitor a respective monitored system;
retrieving, from the memory, a first threshold that is associated with the first parameter and the respective monitored system; and
returning a first system condition alert when the first value has crossed the first threshold.

13. The system of claim 12, wherein:
the first threshold is retrieved from a database that stores a plurality of thresholds for the first parameter, each of the plurality of thresholds being associated with a different type of monitored system; and
the at least one processor is further configured to perform the operations of receiving user input specifying a budget for a system condition of one of monitored system types, and scaling the plurality of thresholds in response to the user input.

14. The system of claim 12, wherein the respective monitored system includes a built-in-test (BIT) subsystem, and returning the first system condition alert includes transmitting the first system condition alert to the BIT subsystem.

15. The system of claim 12, wherein the respective monitored system includes a munition that is part of a plurality of munitions that are stored in a same case or pallet, wherein the at least one processor is further configured to perform the operation of changing an availability rating for the plurality of munitions when the first value has crossed the first threshold, and leaving the availability rating unchanged when the first value has not crossed the first threshold.

16. A method, comprising:
obtaining a mission identifier, the mission identifier specifying a mission for a platform that is arranged to carry a monitored system; and
selecting a threshold value for a threshold, the threshold corresponding to a monitored parameter of the monitored system, the threshold value being selected based on the mission identifier; and
configuring at least one of a notification device and a data assessment system to generate an alert when the monitored parameter has crossed the threshold value.

17. The method of claim 16, wherein selecting the threshold value includes selecting a first threshold value when a first mission identifier is received and selecting a second threshold value when a second mission identifier is received.

18. The method of claim 16, wherein receiving the mission identifier includes at least one of: (i) receiving user input specifying the mission identifier and/or (ii) executing a machine-learning engine to obtain the mission identifier.

19. The method of claim 16, wherein the mission identifier identifies a mission that is selected from a group including a standard operations mission, an emergency operations mission, and a lifecycle maintenance mission, and the platform includes a combat platform.

20. The method of claim 16, further comprising:
receiving a parameter value, the parameter value being received from a monitoring device that is arranged to monitor a respective monitored system, the parameter value identifying at least one of vibration, pressure, temperature, or humidity that is experienced by the respective monitored system;
detecting whether the parameter value has crossed the threshold value,
returning a first maintenance alert when the parameter value has crossed the threshold value.
